(19) United States
(12) Patent Application Publication
(10) Pub. No.: US 2022/0254476 A1
(43) Pub. Date: Aug. 11, 2022

(54) Method And Apparatus For Intelligently Scheduling Surgical Procedures

(71) Applicant: Slim Souissi, San Diego, CA (US)

(72) Inventor: Slim Souissi, San Diego, CA (US)

(73) Assignee: Ospitek, Inc., San Diego, CA (US)

(21) Appl. No.: 17/072,614

(22) Filed: Oct. 16, 2020

(51) Int. Cl. G16H 40/20; G16H 10/60

(52) U.S. Cl. CPC G16H 40/20; G16H 10/60

(57) ABSTRACT

A method for estimating a duration of at least one surgical procedure based on learning from accumulated historical data is provided. The method comprises gathering historical patient data and hospital personnel performance data. The method also comprises analyzing the historical data and the performance data and learning about surgical procedure durations from the analysis. The method also comprises estimating a duration of a surgical procedure or a combination of surgical procedures based on the analysis and learned procedure duration information.

### FIELD OF THE INVENTION

The present disclosure is in the field of hospital and surgical center management. More particularly, the present disclosure provides systems and method of intelligently scheduling surgical procedures based on a plurality of often dynamically changing factors.

### BACKGROUND

Scheduling surgical procedures for patients depends on many parameters such as availability of the surgeon performing the procedure as well as the medical staff supporting surgery, the availability of the operating room and any special equipment needed for the procedure. Scheduling the duration of the procedure is typically performed intuitively based on the experience of the scheduler and feedback from the physician. In general, schedulers use rules of thumbs when assigning a duration for a given procedure. A plastic surgery procedure may be assigned a one-hour slot while a knee replacement procedure is given 90 minutes.

Sometimes the physician dictates the duration if the procedure complexity is unknown to the scheduler or if the patient health condition warrants extending the duration. Schedulers typically err on the conservative side when scheduling procedures. Schedulers wish to avoid mistakes that cause the surgeon to be pressured to finish on time or mistakes in scheduling too many patients for procedures or making misjudgments that cause patients to wait too long.

Scheduling procedures is complex balancing act that involves medical and personnel parameters. When performed by a human, it is conservatively estimated and suboptimal. A need therefore exists for more accurate prediction of procedure duration and with high confidence. Such better prediction may allow for a better utilization of the operating rooms, surgeon time and contributes to minimizing patient wait time.

### BRIEF DESCRIPTION OF THE FIGURES

FIG. 1 through FIG. 9 are diagrams of a system of intelligently scheduling surgical procedures according to embodiments of the present disclosure.

### DETAILED DESCRIPTION

Systems and methods described herein provide for estimating an entire patient journey as patients move from the waiting room, to pre-Op, to an operating room, and to the recovery room. Estimating the duration of the patient journey and every step from registration to discharge has many benefits:

a. It allows a surgical center to operate more efficiently by optimizing the process of scheduling procedures and assigning staff and equipment to these procedures.

b. It allows schedulers to allocate procedure times on the schedule with high accuracy. Wait time for patients is therefore reduced. Unnecessary costly crowding of the medical facility is also reduced.

c. Good estimation also improves patient satisfaction by keeping patient loved ones informed in real time about the status of the patient from the moment they register until they are discharged.

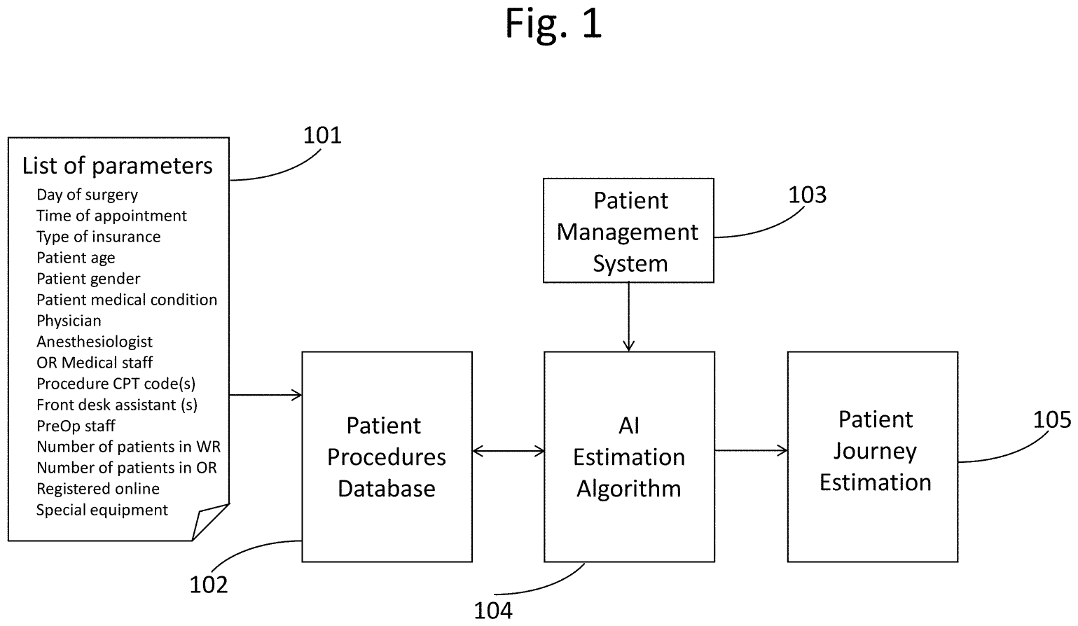

FIG. 1 illustrates an intelligent system for estimating duration of surgical procedures. A main component (104) is an estimation algorithm that is fed a list of parameters (101). The parameters (101) are as follows:

Day of surgery: The day of surgery may have a correlation to the duration of surgery primarily because of human behavior. For example Friday may be more productive than Mondays because staff is motivated to finish on time and not delay the start of their weekend as opposed to Monday which could be more crowded with patients and potential late arrivals of physicians or medical staff.

Time of appointment: Time of day of appointment may be relevant to the duration of surgeries. Surgeons typically want their more difficult surgeries early in the day to assure that by the end of the day before the facility is about to close, the patient has recovered and ready to be discharged. This gives surgeons the necessary time to care for those patients and attempt to get them discharged by late afternoon instead of keeping them overnight.

Type of insurance: This could cause delays in the waiting room if approvals take longer. It could also have an impact on type of equipment used or implant parts that could cause a procedure to last longer

Patient overall medical condition: age and gender may affect the duration of the procedure. A younger healthier patient usually takes less time during the anesthesia process and the recovery process. Younger patients also take less time during surgery because they may not have existing medical conditions that may require additional time-consuming precautions.

Anesthesiologist: this doctor has an important role in the duration of the procedure and the overall duration of the patient journey. Anesthesiologist issues include: Does he/she arrive to the facility on time? Does he/she respond quickly to meeting the patient when the patient is ready for the anesthesia interview? Is the anesthesiologist experienced with drugs type and quantities for difficult procedures that could impact the time it takes to sedate a patient or wake them up?

Medical staff: the experience and discipline of waiting room, preOp, OR and recovery room staff has a role in the duration of the procedure and patient journey. Questions include: Can they multitask? Do they have the experience to deal best with other peers to ensure smooth working environment? Have they worked enough with surgeons and anesthesiologists to anticipate their needs? Can they foresee potential issues and proactively avoid them? Is the person at front desk a trainee or an experienced assistant?

Current Procedural Terminology (CPT) codes: These codes describe medical, surgical, and diagnostic services. CPT codes communicate uniform information about medical services and procedures. Similar procedures tend to take similar amounts of time to be performed. An Esophagoscopy (CPT 43191-43232) may take only 20 to 30 minutes while an 27130 Arthroplasty (CPT 27130) may take 120 minutes.

Number of patients at a given location: The larger the number of patients at a given location, the longer it takes to process these patients. For example, more patients in the preOp usually requires more staff and takes more time to prepare for surgery. More patients in the waiting room increases the time to get patients checked in.

Special equipment: The use of special equipment such as robots can help reduce the duration of surgery.

Online registration of patients reduces their wait time because less time is spent filling out forms and responding to potential questions from medical staff when the patient has unexpected medical conditions.

Patient procedures database (102) contains procedure (CPT codes) and associated attributes from list (101) and durations. Each type of surgery performed is listed along with attributes and normal surgery duration. Also described are various steps of the surgery including the start of intubation, the start of time out, the start of the procedure, the start of the closing and the start of ex-tubation. The database (102) accumulates collected statistical data about various procedures and patient journeys.

Patient management system (103) consists of a patient tracking system to provide live data about patient location in the surgical center and patient status. Based on patient status and location, the patient journey estimates are continuously updated. Patient journey estimation (105) estimates the duration of steps of the patient journey. Such estimates may be used for scheduling purposes, for live patient journey updates, for workflow optimization at the surgical center, and for performance analytics.

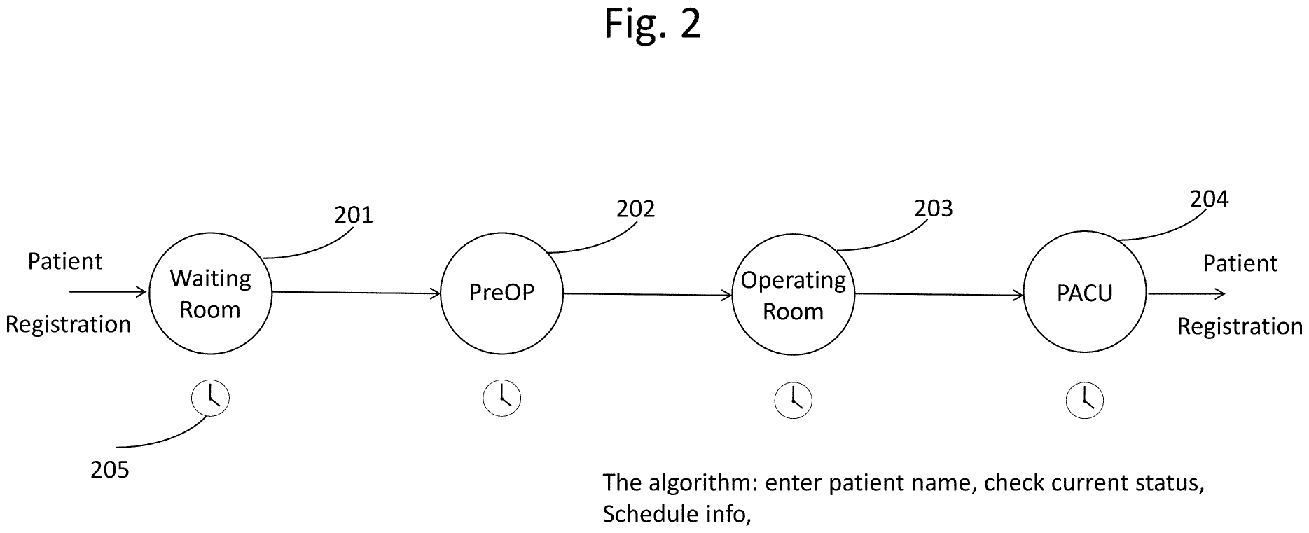

FIG. 2 illustrates a typical patient journey in a hospital or surgical center from registration to discharge. Durations of the journey for each of steps (201), (202), (203) and (204) are timed (205) and stored in a database and/or shared live with the appropriate individuals.

The journey starts with the waiting room (WR) (301). Test (307) is performed to determine if the patient has moved or her/his status has changed. If the answer is “No” then the estimate is not corrected. The original estimate made by (104) is maintained. If the answer is “Yes,” then a new estimate is calculated. For example if the estimated waiting room time is 20 minutes and the patient is still in the waiting room after 23 minutes then the estimate is updated to 23 minutes and the estimate continues to increase until the patient is detected in preOp.

On the other hand, if the estimated waiting room time is 20 minutes and the patient status changes to “ready to move to preOp” after merely five minutes from the arrival of the patient to the waiting room, then the estimated wait time in the waiting room is corrected to 15 minutes. It takes only ten minutes for a patient to be moved from the waiting room to the pre-operative room once the patient is marked as “ready to move to preOp”. A similar process may be used to estimate and refine time estimates when the patient is at the preOp (303), the OR (304) or the PACU (306).

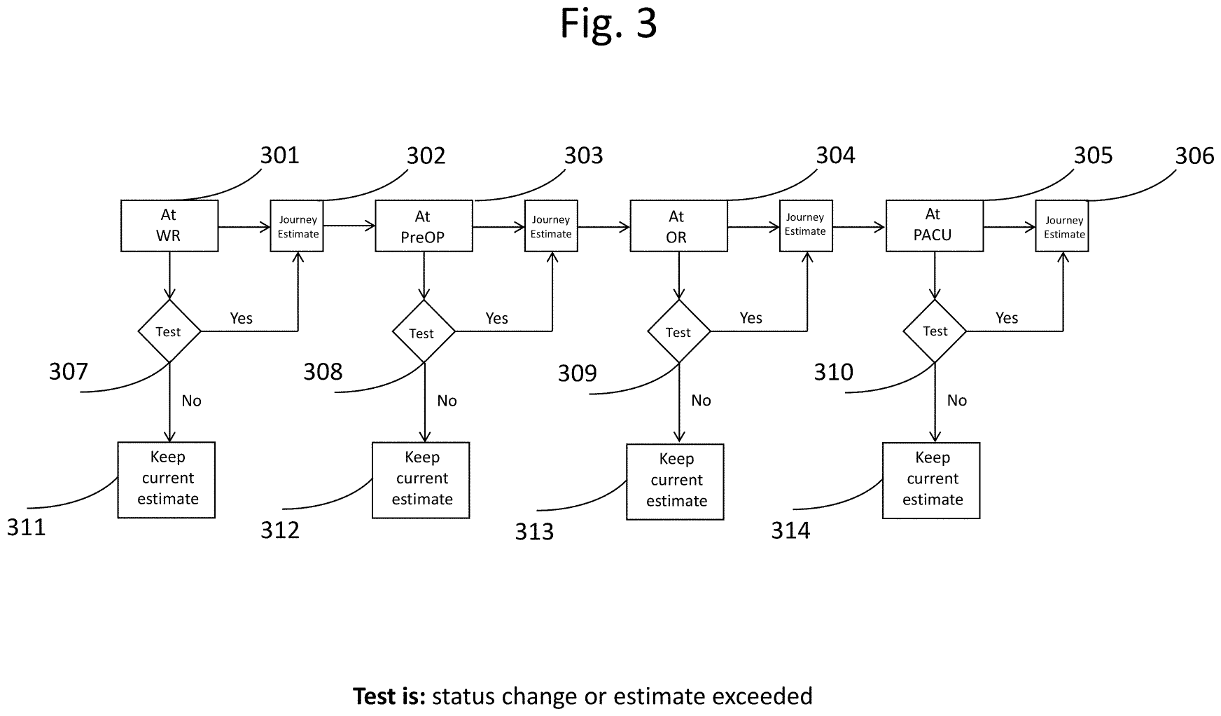

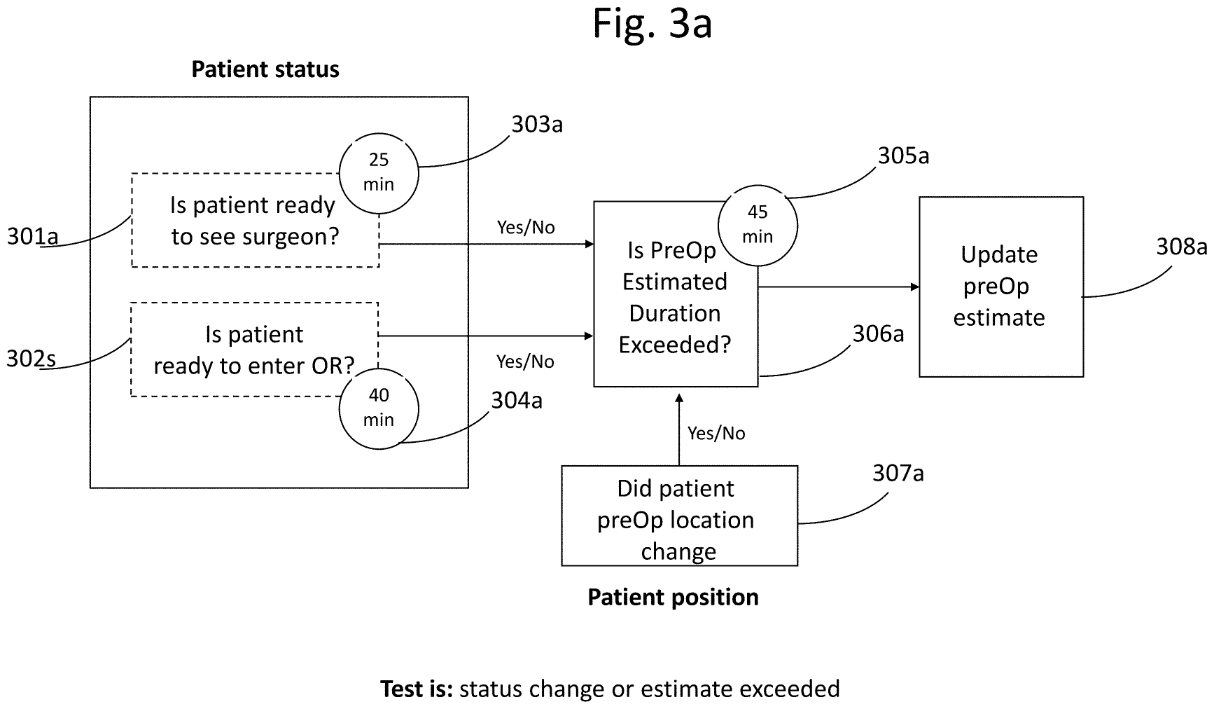

Refinement of estimates may be triggered by patient location or change of status. FIG. 3 illustrates an example of when the patient is in the PreOp room. For this particular patient visit, it is estimated by (104) that the duration of the patient stay at the preOp is 45 minutes (305a). The time it takes for the patient to be ready to see the surgeon is 25 minutes (303a) from the moment she/he enters the preOp. The time it takes for the patient to be ready to move to the operating room is 40 minutes (304a) from the moment she/he enters the preOp.

Estimates of time the patient spends in the preOp is continuously refined based on (i) patient status change: If patient ready for surgeon status exceeds 25 minutes, then the preOp duration estimate is updated accordingly by (308a). If patient status of ready to move to the operating room exceeds 40 minutes, then the preOp duration estimate is updated accordingly and/or (ii) patient location: If patient PreOp location did not change after 45 minutes from entering the preOp, the the preOp duration estimate is updated accordingly.

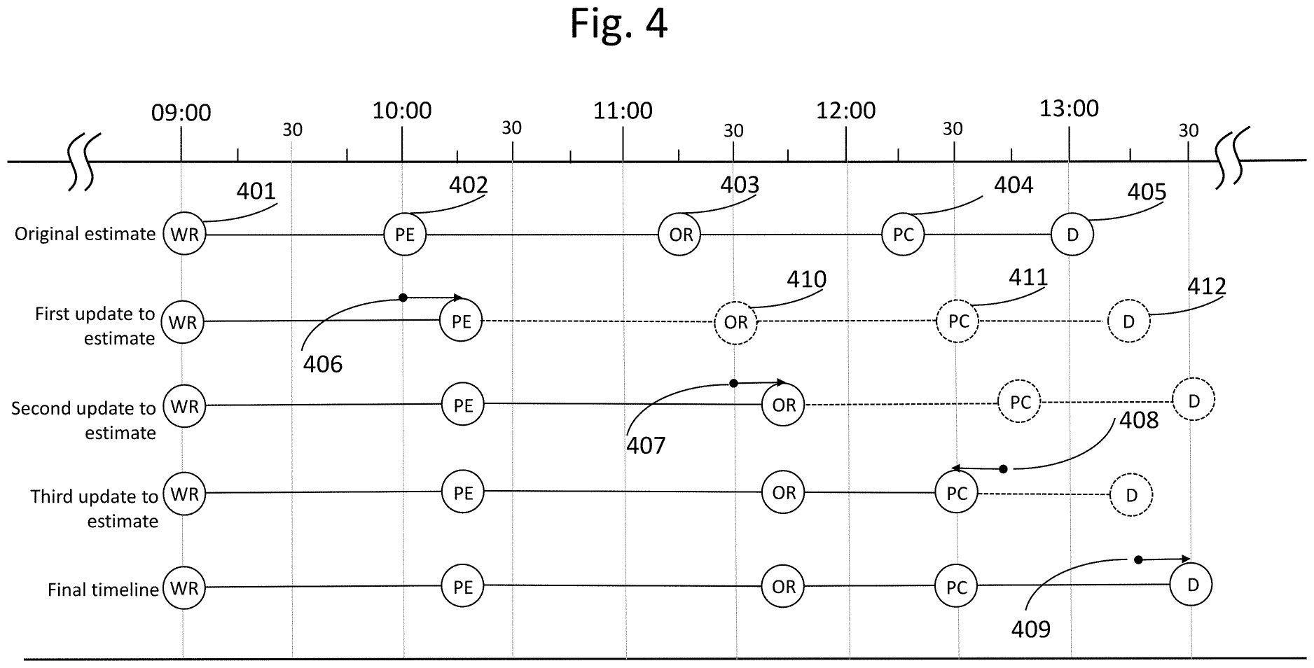

FIG. 4 illustrates the patient journey refinement process. The original estimated performed by (104) is illustrated by steps (401), (402), (403), (404) and (405).

First update to estimate: The patient was delayed by 15 minutes in the preOp and that is shown by (406). This caused an update of the estimated time in OR (410) and recovery (411) and discharge (412) to all be pushed out by 15 minutes.

Second update to estimate: The patient entered the OR late by 15 minutes as shown by (407). This also caused an additional push estimates for PACU and discharge by an additional 15 minutes.

Third update to estimate: The patient stay surgery was lower than originally estimated. This caused pulling in the estimated entry time to PACU by (408).

The final timeline shows that the stay in PACU took longer than estimated by (409). The final timeline including all patient locations and status changes is saved and stored in database (102) to allow for future analytics as well serve as data available for estimation algorithms to build statistics and intelligence for future estimates. A larger the data pool may support better estimates.

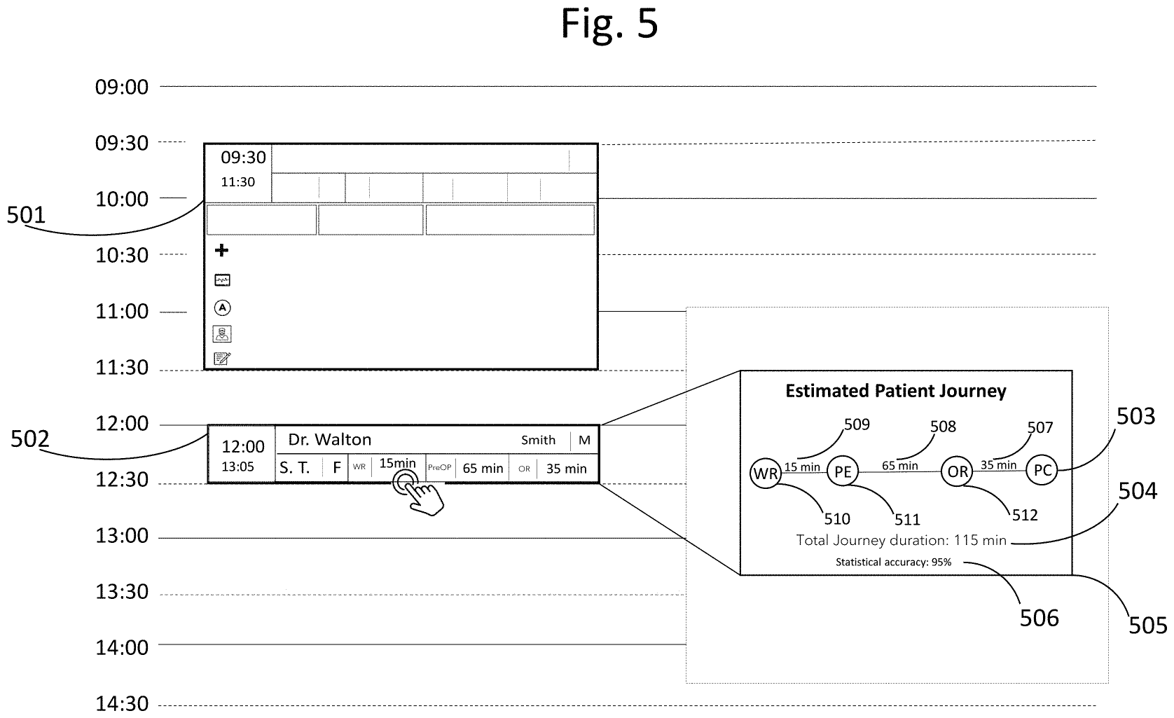

FIG. 5 is an illustration of how schedulers may use systems and methods provided herein to increase efficiency in scheduling procedures and more accurately estimate the booked period for surgery. When the scheduler enters a new procedure (502), the scheduling algorithms make use of patient and procedure information (101) to estimate the patient journey (505). In this case, the estimated total journey is 115 minutes (504) with a 95% accuracy (506). Details of the journey are shown by (509), (508) and (507). The estimated procedure duration is 65 minutes. The scheduler books the procedure to start at 12:00 and end at 13:05 as shown by (502).

This information may be valuable for surgical center staff to plan their activities for other surgeries. The information may also be valuable for a patient's family to know ahead of time when they need to drop off and pick up their loved ones.

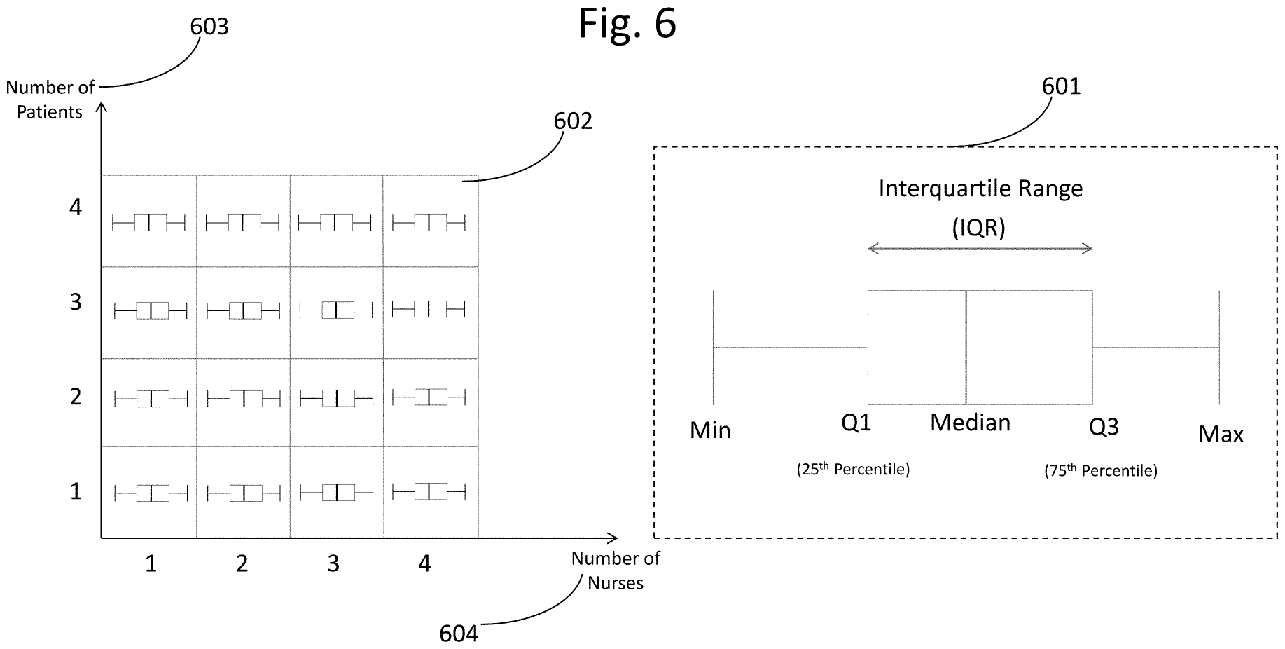

FIG. 6 is a two-dimensional graph that captures a statistical date for patient wait time in the preOp or the recovery room. A larger number of nurses (604) in the preOp may lead to improvements in quality of service and patient wait time. In general, the larger the number of patients (603), the longer the wait time. Quadrant (602) shows statistical data for a case when four patients are present in the preOp and four nurses are servicing them. (601) illustrates the type of data used by the intelligent estimation algorithm (104) to estimate patient service wait time. Information such as the minimum, median and maximum are collected in the database (102). Various percentile statistics such as Q1 and Q2 are also collected to estimate the level of confidence in the estimates.

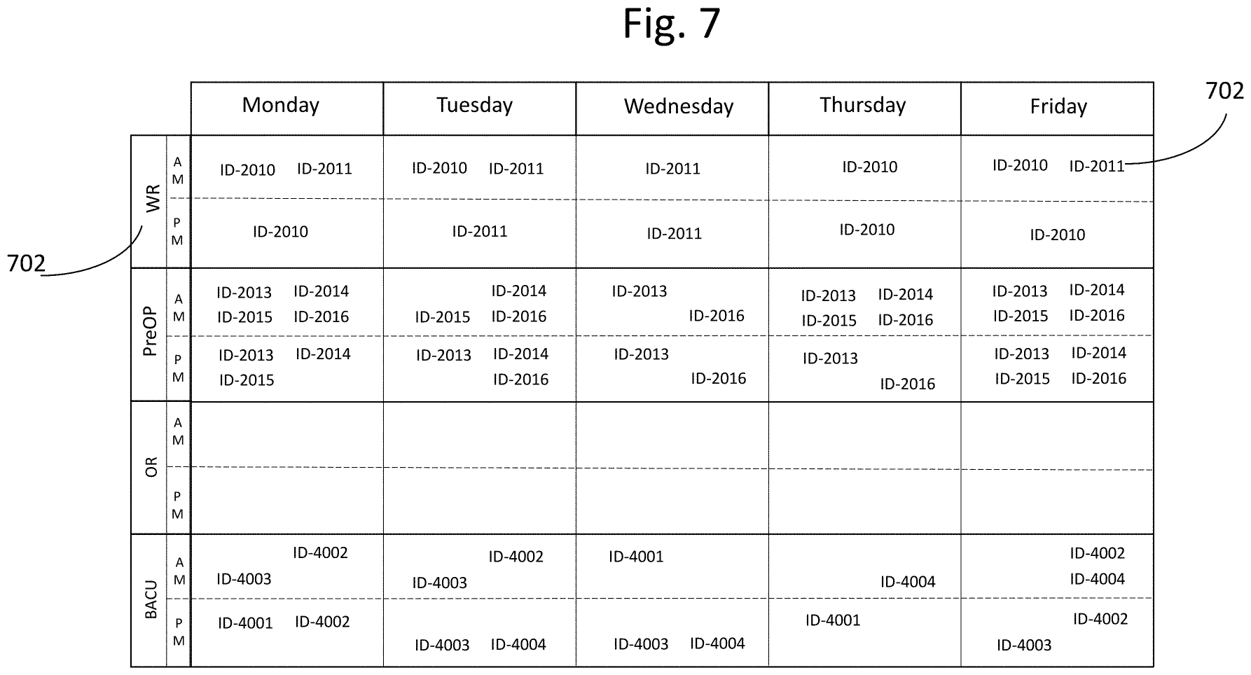

FIG. 7 shows an example of staffing schedule for a surgical center depending on the location (702). The staff ID (702) is shown in the quadrant where the subject staff member is expected to report to work. This type of schedule is used by the intelligent estimation algorithm (104) to estimate the patient wait time various locations of the surgical center.

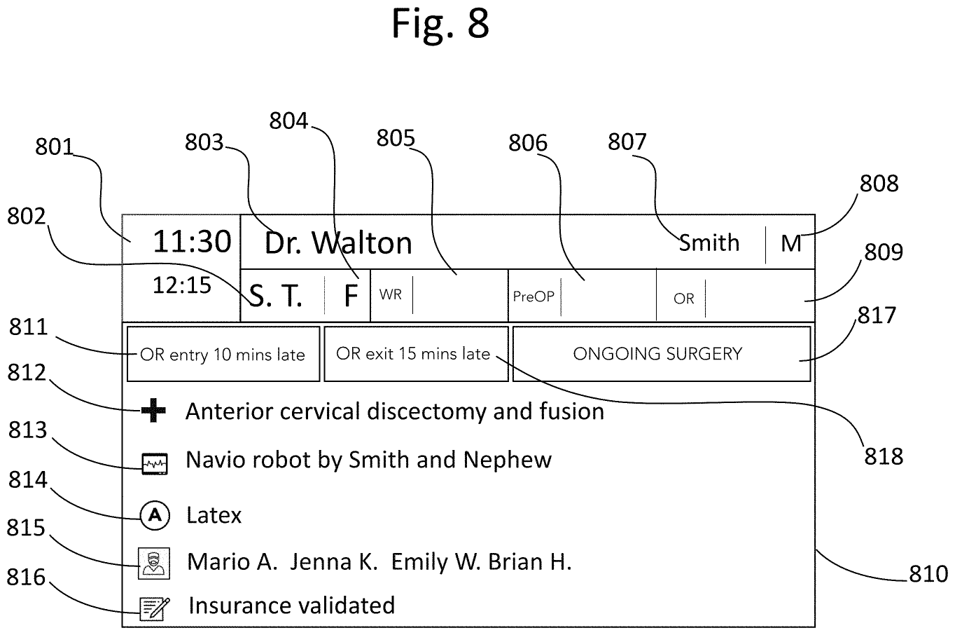

FIG. 8 shows a detailed view of information required by the surgical center to identify a procedure on scheduler board (206). This information includes (801) start time and end time of the procedure, (802) patient initials in compliance with Health Insurance Portability and Accountability Act (HIPPA) requirements, (803) physician name, (804) patient gender, (805) duration of patient stay at the waiting room, (806) duration of patient stay at the preOp, (809) duration of patient stay at the OR, (807) anesthesiologist name, (808) type of anesthesia, (811) label to visualize late OR entrance, (818) label to visualize late OR exit, (817) label to visualize surgery status while patient is in the OR. (812) shows the procedure name, (813) shows the type of special equipment needed for the procedure, (814) shows the type allergies, (815) shows the list of medical staff attending the procedure, (816) shows general information about the procedure that could relate to insurance or administrative data. This information, along with historical statistical data from (102) is used by the intelligent estimation algorithm (104) to estimate patient wait time at various location of the surgical center.

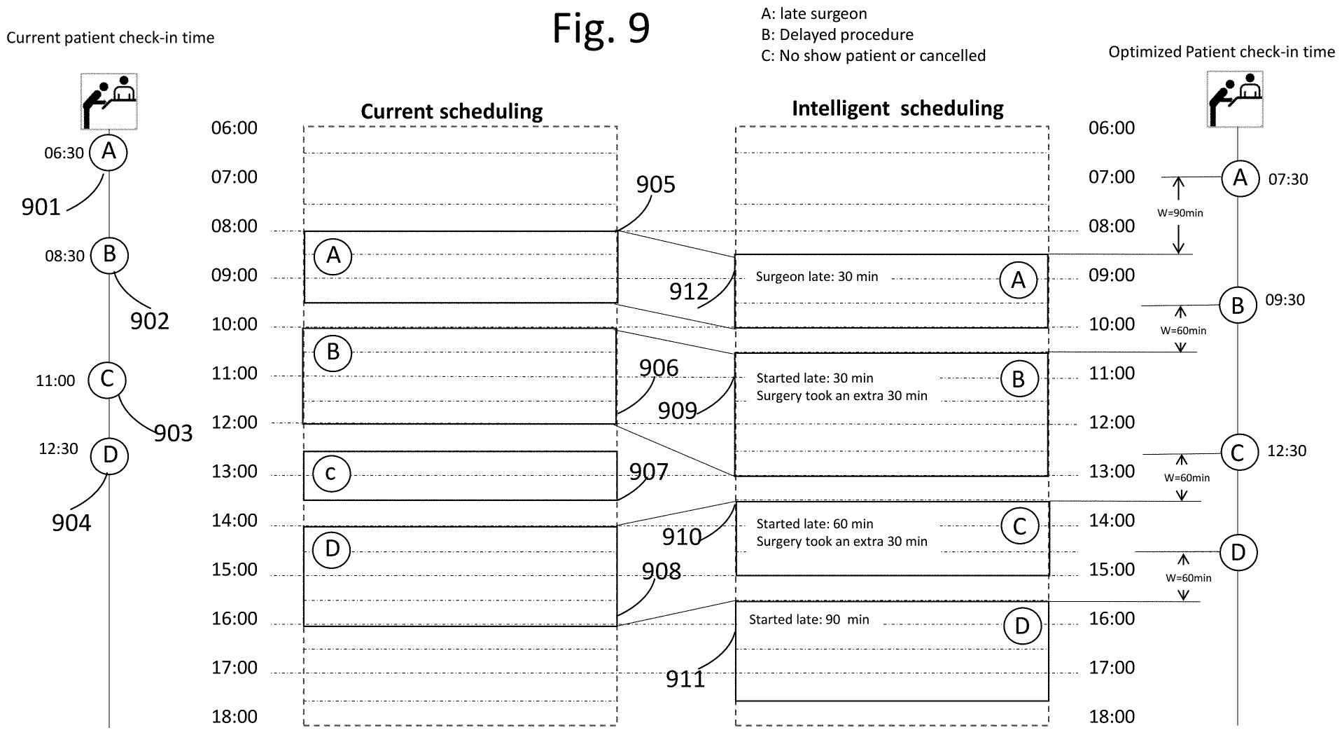

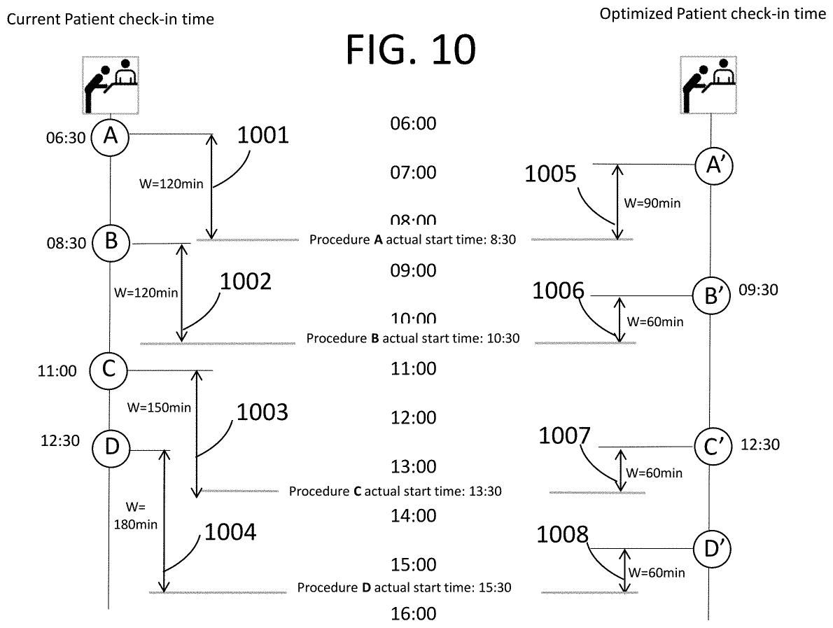

FIG. 9 provides an example of efficiencies that may be provided by systems and methods provided herein. Under previous implementations, patients A (901), B (902), C (903) and D (904) are scheduled to arrive at the waiting room respectively at 6:30, 8:30, 11:00 and 12:30. Their respective scheduled procedures (905), (906), (907) and (908) are shown on the schedule with their planned start time and duration. Because operation of a surgical center may be dynamic, changes to a schedule may occur because of delayed procedures and delayed arrival of patients, staff members and physicians. The intelligent scheduling system promotes monitoring such changes and live optimization of the schedule.

For example, a procedure for patient A has changed to (912). It is delayed by 30 minutes because the physician was late. The new start time is pushed out from 8:00 to 8:30. The patient arrival time which is generally scheduled 90 minutes before the start of surgery is now set to only 60 minutes because of the higher confidence in the patient journey estimate provided by the intelligent scheduling system. Patient A is now scheduled to arrive at 7:30 instead of 6:30 despite the delayed surgeon arrival. The wait time for patient A has improved from 120 minutes (1001) to 60 minutes (1005) as illustrated by FIG. 10.

A procedure for Patient B began late by 30 minutes because of the delayed start of the previous procedure and took 30 minutes longer than its scheduled duration. In this case, Patient B, who was supposed to check in at the surgical center at 8:30, would start his or her procedure at 10:30 and would wait 120 minutes (1002). By using intelligent scheduling as provided herein, Patient B is notified to arrive at 9:30 instead and his/her waiting time is improved to 50 minutes (106).

A procedure for Patient C began late by 60 minutes because of the delayed previous procedure and took 30 minutes longer than what it was scheduled for. In the case, Patient C who was scheduled to check in at the surgical center at 11:00 would start his/her procedure at 13:30 and would wait 150 minutes (1003). By using intelligent scheduling, Patient C is notified to arrive at 12:30 (not 11:00) and his/her waiting time is improved to 60 minutes (107).

A procedure for Patient D began late by 90 minutes because of the delayed previous procedure. In the case, Patient D, who was supposed to check in at the surgical center at 12:30, would start his/her procedure at 15:30 and would wait 180 minutes (1004). By using intelligent scheduling provided herein, Patient D is notified to arrive at 12:30 (not 11:00) and his/her waiting time is improved to 60 minutes (108).

The case described above of four scheduled patients demonstrates that the aggregate wait time has improved from (120+120+150+180=570 mins) to (90+60+60+60=270 mins). This represents an improvement of 300 minutes of patient wait time. Such an improvement is beneficial to surgical centers. It contributes to better patient satisfaction because patients are not frustrated anxiously waiting for their surgeries. It also improves with efficient use of staff by minimizing patient crowding within the surgical center. A lower number of patients at various times and locations of the center may reduce costs of providing care and elevate the quality of care. When the number of patients in the waiting room, preOp, or PACU is reduced, the nurse staffing load may be reduced. Cost efficiencies may consequently be realized.

What is claimed is:

1. A method for estimating a duration of at least one surgical procedure based on learning from accumulated historical data, comprising:
gathering historical patient data and hospital personnel performance data;
analyzing the historical data and the performance data and learning about surgical procedure durations from the analysis; and
estimating a duration of a surgical procedure or a combination of surgical procedures based on the analysis and learned procedure duration information.

2. The method of claim 1, wherein the historical data includes patient health information. The method of claim 1, wherein the historical data includes doctor or medical staff historical performance.

4. The method of claim 1 wherein the historical data includes a factor representative of how busy the healthcare facility is.

5. The method of claim 1 wherein the estimate of the duration depends on the type of procedure performed.

6. A method for estimating the duration of a patient journey within healthcare facility, comprising adding:
an estimated time spent by a patient at (i) a waiting room to
an estimated time spent by the patient at (ii) a pre-operative room to
an estimated time spent by the patient at (iii) an operating room to
an estimated time spent by the patient at (iv) a recovery room; and
publishing the sum of the estimated times as a duration of a journey of the patient.

7. The method of claim 6, wherein the estimated time in the waiting room (or pre-operative room or recovery room) is calculated based on the historical performance of one or more staff members working at the waiting room (or pre-operative room or recovery room).

8. The method of claim 7, wherein the estimated time is equal to a percentile value of historical performance times of one or more staff members working at the waiting room (or pre-operative room or recovery room).

9. The method of claim 7, wherein the percentile value depends on the number of staff working at the waiting room (or pre-operative room or recovery room). and the number of patients in the present at the waiting room (or pre-operative room or recovery room).

10. The method of claim 6, wherein the estimated time in the recovery room is calculated based on the historical performance of the staff working at the waiting room.

11. The method of claim 6, wherein the estimated duration of the patient journey is updated in real time reflecting changes in the patient status or location within the surgical center.

12. The method of claim 11, wherein the updated estimates are communicated in real time to the patient loved ones via a phone application, a web page or a text message.

13. An interactive patient procedure scheduling system that estimates durations of surgical procedures based on learning from accumulated historical data and communicates to a scheduler an estimated duration of a procedure for booking said estimate on a surgical schedule board, comprising at least one application that:
adds estimates of time spent by a patient
(i) at a waiting room,
(ii) at a pre-operative room,
(iii) at an operating room, and
(iv) at a recovery room, and
communicates a total of the estimates to a scheduler, the total posted by the scheduler to a surgical schedule board.

14. The system of claim 13, wherein the estimated time in the waiting room (or pre-operative room or recovery room) is calculated based on historical performance of one or more staff members working at the waiting room (or pre-operative room or recovery room).

15. The system of claim 14, wherein the estimated time is equal to percentile value of historical performance times of one or more staff members working at the waiting room (or pre-operative room or recovery room).

16. The system of claim 15, wherein the percentile value depends on the number of staff working at the waiting room (or pre-operative room or recovery room). and the number of patients in the present at the waiting room (or pre-operative room or recovery room).

17. The system of claim 13, wherein the estimated time in the recovery room is calculated based on the historical performance of the staff working at the waiting room.

18. The system of claim 13, wherein an estimated duration of the patient journey is updated in real time reflecting changes in the patient status or location within the surgical center.

19. The system of claim 13, wherein the estimates are updated and communicated in real time to patient loved ones

20. The system of claim 19, wherein the estimates are communicated via at least one of a phone application, a web page or a text message.
